(19) United States
(12) Patent Application Publication
(10) Pub. No.: US 2006/0030828 A1
(43) Pub. Date: Feb. 9, 2006

(54) Absorbent article having a disposal member

(75) Inventors: Hoa La Wilhelm, Appleton, WI (US); Cindy Lou Price, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/911,826

(22) Filed: Aug. 4, 2004

(51) Int. Cl. A61F 13/15

(52) U.S. Cl. 604/359; 604/385.13; 604/385.19

(57) ABSTRACT

An absorbent article is disclosed having a disposal member. The absorbent article includes a cover, a baffle and an absorbent positioned therebetween. The disposal member is configured as a pocket having a fixed portion and an open portion. The fixed portion is secured to at least one of the cover, absorbent or baffle. The disposal member is formed from a material having an odor control substance integrally formed therewith for limiting any malodors exiting the absorbent. The open portion of the pocket is capable of receiving and enclosing a substantial portion of the soiled absorbent article after it has been reduced in overall size for convenient disposal.

### BACKGROUND OF THE INVENTION

For a long time, many users of disposable absorbent articles have desired a discrete means of disposing of their used and soiled absorbent articles. Disposable absorbent articles are articles designed to absorb human exudate, like urine, menses and fecal matter. Examples of such disposable absorbent articles include catamenial products such as feminine pantyliners, sanitary pads and napkins, tampons, adult incontinent pads, briefs and undergarments. Infant diapers, child training pants and other disposable absorbent articles could also benefit from having an integral disposal member that will permit discrete disposal. Disposable absorbent articles are primarily designed for a single use, after which they are discarded usually into a toilet pail, trash receptacle or flushed down a toilet. However, storage in a toilet pail located in a bathroom or in some other trash receptacle rapidly results in the development of disagreeable odors, such as ammonia. Disposal of a soiled feminine pad or incontinence garment at a friend's house or in a company's restroom can be awkward and sometimes embarrassing. The emission of malodors from a soiled discarded absorbent product or the possibility of having a coworker notice that you are having your period can be embarrassing. Worst yet, it can be psychologically traumatizing to an older person who needs to dispose of an incontinent article while a relative, friend or coworker is nearby and knowing that when the article is discarded, the other person will become aware of the fact that the older adult is physically required to use such products. This traumatic experience can also occur in an older adult who has just started to use such incontinent undergarments. On one of their first trips away from home, they find themselves in need of disposing of a soiled incontinent undergarment and would like to be able to do so in a secretive and discreet manner. By doing so, they can refrain from having to disclose the fact that they are now incontinent.

Therefore, there is a need for a disposable absorbent article which has a disposal member which will permit the soiled absorbent article to be wrapped and handled in a sanitary manner and will limit the emission of malodors. The soiled article can be temporarily stored in a purse, coat pocket or shopping bag until the user can secretively and discreetly dispose of the article.

### SUMMARY OF THE INVENTION

Briefly, this invention relates to an absorbent article having a disposal member. The absorbent article includes a cover, a baffle and an absorbent positioned therebetween. The disposal member is configured as a pocket having a fixed portion and an open portion. The fixed portion is secured to at least one of the cover, absorbent or baffle. The disposal member is formed from a material having an odor control substance integrally formed therewith for limiting any malodors exiting the absorbent. The open portion of the pocket is capable of receiving and enclosing a substantial portion of the soiled absorbent article after it has been reduced in overall size for convenient disposal.

### BRIEF DESCRIPTION OF THE DRAWINGS

FIG. 1 is a bottom view of an absorbent article having a disposal member formed from a material having an odor control substance integrally formed therewith.

FIG. 2 is a cross-sectional view of FIG. 1 taken along line 2-2 showing the various layers which form the absorbent article.

FIG. 3 is a cross-sectional view of the absorbent article shown in FIG. 2 taken along line 3-3 depicting an odor control substance integrally formed in the material forming the disposal member.

FIG. 4 is a perspective view of the absorbent article shown in FIG. 1 when the absorbent article is partially rolled up.

FIG. 5 is a perspective view of the absorbent article shown in FIG. 1 when the disposal member is inverted and extended over and around the rolled up absorbent article.

FIG. 6 is a side view of the absorbent article shown in FIG. 1 when the absorbent article is folded in half and the disposal member is inverted and extended over and around a portion of the absorbent article.

FIG. 7 is a top view of an alternative embodiment showing an absorbent article having a pair of disposal members each formed from a material having an odor control substance integrally formed therewith.

FIG. 8 is a cross-sectional view of FIG. 7 taken along line 8-8 showing the various layers which form the absorbent article.

FIG. 9 is a cross-sectional view of the absorbent article shown in FIG. 7 taken along line 9-9 showing the disposal member formed with pleats so that it can easily expand to form a pocket.

FIG. 10 is a top view of another embodiment of an absorbent article having a disposal member formed from a material having an odor control substance printed thereon.

FIG. 11 is a top view of still another embodiment of an absorbent article having a pair of disposal members each extending beyond the opposite ends of the absorbent article.

FIG. 12 is a top view of still another embodiment of an absorbent article having a disposal member with a V-shaped opening formed therein.

FIG. 13 is a bottom view of another embodiment of an absorbent article having a disposal member formed completely over the baffle and having an odor control substance integrally formed therewith.

FIG. 14 is a cross-sectional view of the absorbent article shown in FIG. 13 taken along line 14-14 showing the disposal member formed with an expandable pocket.

FIG. 15 is a perspective view of the absorbent article shown in FIG. 13 when the absorbent article is partially rolled up.

FIG. 16 is a perspective view of the absorbent article shown in FIG. 13 when the disposal member is inverted and extended over and around the rolled up absorbent article.

### DETAILED DESCRIPTION

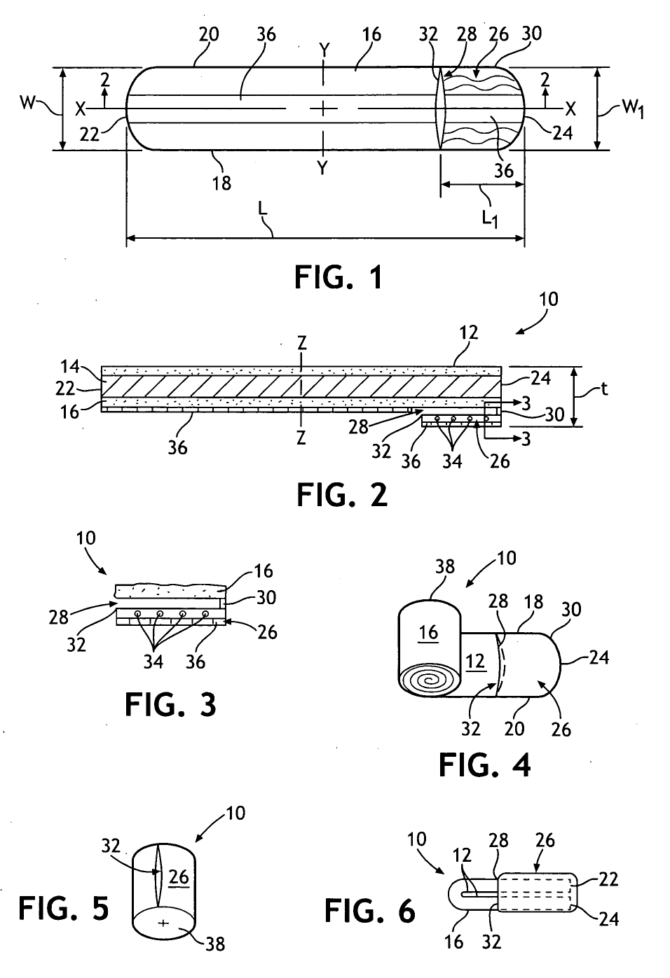

Referring to FIGS. 1 and 2, an absorbent article 10 is shown for absorbing human exudate. The human exudate can include urine, menses, perspiration and other bodily fluids, as well as semi-solid and solid excrement. The absorbent article 10 can be constructed as a feminine pantyliner, a sanitary napkin, an adult incontinent pad, brief or undergarment, an infant diaper, a child training pant, a menstrual pant, etc. The absorbent article 10 can be a disposable article or be a reusable article. By “disposable” it is meant an article that is designed for a single or temporary use and is meant to be discarded after being used once. By “reusable” it is meant an article that is designed to be laundered, washed, dry cleaned, etc. for reuse multiple times, such as a typical cotton underpants.

Still referring to FIGS. 1 and 2, the absorbent article 10 includes a cover 12, an absorbent 14 and an optional baffle 16. When the absorbent article 10 is constructed from all three layers, the absorbent 14 is positioned between the cover 12 and the baffle 16. The layers 12, 14 and 16 can be directly or indirectly secured to an adjacent layer or to the other layer by a construction adhesive, not shown. The construction adhesive can be either a hot or cold melt adhesive. When the absorbent article 10 is constructed from only the cover 12 and the absorbent 14, the absorbent 14 is positioned adjacent to the cover 12 and may be directly or indirectly secured thereto.

The cover 12, also sometimes referred to as a bodyside liner should be liquid permeable or liquid pervious so as to allow various body fluids to quickly and easily pass down through it into the absorbent 14. The cover 12 may also be liquid-impermeable prior to use and become liquid permeable during use. The liquid permeable cover 12 is located nearest to the human body, adjacent to the skin of the user, when the absorbent article 10 is worn. Alternatively, the cover 12 can include one or more liquid permeable regions and one or more liquid-impermeable regions. The liquid permeable region(s) formed in the cover 12 should be arranged such that when the absorbent article 10 is worn adjacent to the crotch region of a human body, the liquid permeable region(s) will directly receive body fluids while the liquid-impermeable region(s) can be spaced apart from the area of fluid insult. The cover 12 can be formed from a woven or non-woven material. Normally, the cover 12 is a thin web of material formed from natural or synthetic fibers, with or without apertures formed therein. A spunbond and a bonded carded web are two exemplary materials that work well as a liquid permeable cover 12. “Spunbond” is manufactured and sold commercially by Kimberly-Clark Corporation having an office at 401 North Lake Street, Neenah, Wis. 54956.

The absorbent 14 can be formed from natural or synthetic materials. The absorbent 14 can be made from cellulosic fibers, wood pulp, textile fibers or other absorbent materials known to those skilled in the art. Superabsorbents, in solid form and in the shape of small particles, granules, flakes, etc., can be mixed, added, attached, printed or otherwise secured to the absorbent material to increase the absorbent capacity of the absorbent 14. Superabsorbents and/or the absorbent 14 can also be formed by in-situ polymerization directly in the structure of the absorbent 14 or on another substrate such as the cover 12 or the baffle 16. If the absorbent article 10 is constructed without a baffle 16, the absorbent 14 can be treated, such as by an agent, coating or surfactant, that will limit or prevent a fluid from passing therethrough. Either the lower surface or a lower portion of the thickness of the absorbent 14 can be so treated. The lower surface of the absorbent 14 can be rendered liquid-impermeable by coating, printing or treating the absorbent 14 with a material that will prevent fluid from passing therethrough. A portion of the absorbent 14 can also be made liquid-impermeable by the manufacturing process that is used to construct the absorbent article 10. For example, the absorbent 14 can be run through a pair of nip rolls such that its lower surface or a lower portion thereof is densified and thereby will prevent body fluid from readily passing therethrough. Other ways of making at least the lower surface of the absorbent 14 liquid-impermeable are known to those skilled in the art.

The baffle 16, also sometimes referred to as the garment facing layer, should be liquid-impermeable, at least a region thereof, so as to prevent body fluids from passing therethrough. The baffle 16 may be liquid permeable initially and become liquid-impermeable during use. The baffle 16 can be both liquid-impermeable and air and vapor-impermeable. Alternatively, the baffle 16 can be liquid-impermeable while at the same time being air and vapor permeable, if desired. An air and vapor permeable material is sometimes referred to as a breathable material. In this case, body fluids will be prevented from passing through the baffle 16 while air and vapors are allowed to pass therethrough. The baffle 16 is located on the exterior of the absorbent article 10, away from the skin of the user. The liquid-impermeable baffle 16 is designed to slow down and desirably restrict the body fluids from penetrating and passing therethrough and thereby assure that the user's outer clothing will not become soiled. Desirably, the baffle 16 has a soft feel so as not to chafe or scrape the inner thighs of the wearer of the absorbent article 10. The baffle 16 can be formed from natural or synthetic fibers. The baffle 16 can be formed from a material that is not noisy when squeezed or wrinkled so that the wearing of the absorbent article 10 remains discreet. The baffle 16 can further be formed from a single material or from two or more materials, as in the form of a laminate. Examples of various materials that can be used to construct the baffle 16 include a polyolefin, such as polypropylene, polyethylene or a blend thereof, a thermoplastic material, a thermoplastic film, a polymeric film, a liquid-impermeable layer bonded to a spunbond layer, etc. Other materials known to those skilled in the art can also be utilized. When the baffle 16 is a laminate containing two or more layers, only one of the layers needs to be liquid-impermeable.

The absorbent article 10 is depicted in FIGS. 1 and 2 as an elongated pad having a length L, a width W and a thickness t. The dimensions of the length L, width W and thickness t can vary to suit one's particular needs. The absorbent article 10 also has a longitudinal central axis X-X, a central transverse axis Y-Y and a vertical axis Z-Z. The absorbent article 10 also has a pair of side walls 18 and 20 and a pair of spaced apart ends 22 and 24. The first end 22 is formed at least about 4 inches (about 100 millimeters) away from the second end 24. It should be noted that although the absorbent article 10 is depicted as an absorbent pad in FIGS. 1 and 2, it could be constructed in a variety of geometrical shapes or configurations to include the shape of a diaper, training pant, menstrual pant, adult incontinent brief or undergarment wherein it has a waist opening and a pair of leg openings.

Referring now to FIGS. 1-3, the absorbent article 10 further includes a disposal member 26 configured as a pocket 28 having a fixed portion 30 and an open portion 32. By “pocket” it is meant a small, flat pouch formed into the absorbent article 10 and used to encompass or enclose at least a portion of the remaining material forming the absorbent article 10, once that remaining material has been reduced in size. The pocket 28 can also be viewed as a receptacle or cavity. The disposal member 26 is secured about the second end 24 of the absorbent article 10 and the open portion 32 is aligned perpendicular to the central longitudinal axis X-X of the absorbent article 10. However, it should be mentioned that the disposal member 26 could be situated around the first end 22 if desired and the open portion could be aligned at an angle to the longitudinal axis X-X, if desired. In FIG. 1, the open portion 32 is aligned at 90 degrees to the longitudinal axis X-X. Desirably, the pocket 30 will be able to receive and enclose either a substantial portion of the absorbent article 10 or the entire absorbent article 10. This feature will be explained in more detail shortly.

The fixed portion 30 of the pocket 28 can be a seal, bond, attachment, mechanical connection, etc. that secures the pocket 28 to at least one of the cover 12, the absorbent 14 or the baffle 16. Desirably, the fixed portion 30 is secured to either the cover 12 or to the baffle 16. During manufacturing, the fixed portion 30 can be secured to the cover 12 when the disposal member 26 is situated above the cover 12 in the finished absorbent article 10 and can be secured to the baffle 16 when the disposal member 26 is situated below the baffle 16, as is depicted in FIGS. 1-3. If no baffle 16 is present in the absorbent article 10, the fixed portion 30 can be either secured to the cover 12 or to the absorbent 14. The fixed portion 30 can be secured by an adhesive, an ultrasonic bond, a thermal bond, a heat bond, a pressure bond, a heat and pressure bond, a combination of such bonds, by a mechanical fastener such as thread, snaps, pins, etc., or by other means known to those skilled in the art. The fixed portion 30 can be coterminuous with the outer periphery of the absorbent article 10, as shown, or can be spaced inward of the periphery. In FIG. 1, the fixed portion 30 is aligned with the end 24 and extends along a portion of each of the side walls 18 and 20.

Referring again to FIGS. 1 and 2, the disposal member 26 has a length L₁ and a width W₁, see FIG. 1, and the absorbent article 10 and the disposal member 26 have a combined thickness t, see FIG. 2. The length L, and width W₁ can vary in dimension. However, the length L, of the disposal member 26 should be less than about 50% of the length L of the absorbent article 10. Desirably, the length L₁ of the disposal member 26 should be less than about 40% of the length L of the absorbent article 10. More desirably, the length L₁ of the disposal member 26 should be less than about 30% of the length L of the absorbent article 10. It should be noted that when the disposal member 26 is liquid-impermeable and is initially situated above and overlaps a portion of the bodyside cover 12, the length L₁ of the disposal member 26 should be less than about 25% of the length L of the absorbent article 10 so as not to interfere or hinder the passage of body fluid from the wearer into the absorbent article 10. The width W₁ of the disposal member 26 can be greater than, equal to or less than the width W of the absorbent article 10. In FIG. 1, the width W₁ of the disposal member 26 is shown to be approximately equal to the width W of the absorbent article 10. The thickness of the disposal member 26 can vary from a few millimeters up to about 50 millimeters or more, depending on the construction of the absorbent article 10. The thickness t of the combined absorbent article 10 and disposal member 26 can vary from between about 2 millimeters to about 50 millimeters. Desirably, the thickness t of the combined absorbent article 10 and disposal member 26 will be less than about 40 millimeters.

Still referring to FIGS. 1-3, the disposal member 26 can be the same material that was used to form either the cover 12 or the baffle 16 or it can be a completely different material. The disposal member 26 can be liquid permeable or liquid-impermeable. Desirably, the disposal member 26 is formed from a material that is soft and pliable and can expand or increase in dimension in at least one direction. The disposal member 26 can be formed from woven or non-woven materials. The disposal member 26 can also be formed from elastic or non-elastic materials. Likewise, the disposal member 26 can be formed from an extensible or non-extensible material, a breathable or non-breathable material, a biodegradable or non-biodegradable material, or any combination of the aforementioned materials. The disposal member 26 can also be formed from a material that may or may not be elastically contractible, that is, the material can be stretched but may not be capable of returning to its non-expanded or initial dimension. Desirably, the disposal member 26 is formed from a stretchable and contractible material. The term “resiliently extensible” is used to describe such a stretchable and contractible material. A resiliently extensible material has the properties and characteristics such that it can be stretched or extended when subjected to a tension or pulling force, and when the force is removed, the material will return to substantially its initial or original length. Because of hysteresis, the resiliently extensible material may not be able to recover completely or return 100% from its original pre-stretched dimension. However, a resiliently extensible material will be able to return to at least 80% of its original pre-stretched dimension.

Many elastic materials qualify as being resiliently extensible in that they are capable of being extended and retracted in at least one direction. The disposal member 26 can also be formed from a material that is capable of being stretched and contracted in two or more directions. Desirably, the disposal member 26 is an elastic material that can stretch at least about 70% of its original length in at least one direction. More desirably, the disposal member 26 is an elastic material that can stretch at least about 100% of its original length in at least one direction. Even more desirably, the disposal member 26 is an elastic material that can stretch at least about 100% of its original length in two or more directions.

It should be pointed out that the disposal member 26 can also be made to be expandable or extendible by doubling the material upon itself such as by forming one or more pleats. Other ways of configuring the disposal member 26 such that the material can increase in dimension is to form folds, crepes, wrinkles, etc. therein. The pleats, folds, crepes, wrinkles, etc. can be secured by breakable bonds or they can be unattached from one another. When the material is so overlapped, it is possible for the disposal member 26 to take on a greater dimension once the material is expanded or unfurled. As the material spreads or balloons outward, the volume of the pocket 28 will increase or become bigger.

The disposal member 26 further has an odor control substance 34 integrally formed therein or thereon. The odor control substance 34 can be an agent, an additive or some other substance that is designed to effectively and efficiently reduce and/or eliminate malodors. A malodor is a foul smelling, unpleasant or offensive odor. When an absorbent article is insulted with urine, an unpleasant odor is given off since a byproduct of urine is ammonia (NH₃). The odor control substance 34 can be selected from a group consisting of a: zeolite, activated carbon, clay, molecular sieve, peroxyacid, carbonate, bicarbonate, perfume, starch, superabsorbent or a mixture thereof. Furthermore, the odor control substance 34 can be an antimicrobial agent, a chelating agent or a mixture thereof. In essence, the odor control substance 34 can be any known substance which can minimize, prevent, eliminate, suppress, reduce, subdue or curtail the emission of malodors from the absorbent article 10.

Any odor control agent, antimicrobials or combination thereof, which is known to those skilled in the art, may be used herein. The odor control agents can typically be classified according to the type of odor the agent is intended to combat. Odors may be chemically classified as being acidic, basic or neutral. Alternatively, the odor control agents may be categorized with respect to the mechanism by which the malodor detection is reduced or prevented. For example, odor control agents which chemically react with malodorous compounds or with compounds which produce malodorous degradation products, thereby generating compounds lacking odor or having an odor acceptable to consumers, may also be utilized.

Suitable odor control agents for use herein typically include carbonates (e.g., sodium carbonate), bicarbonates (e.g., sodium bicarbonate), phosphates (e.g., sodium phosphate), sulphates (e.g., zinc and copper sulphates), carboxylic acids such as citric acid, lauric acid, boric acid, adipic acid and maleic acid, activated carbons, clays, molecular sieve, synthetic or natural zeolites, silicas, superabsorbents and starches. One commercially available zeolite is sold by Union Carbide Corporation under the trademark ABSCENTS®.

Suitable odor control agents also include chelating agents and may be selected from cyclodextrin, modified cyclodextrin, amino carboxylates such as ethylenediamine-tetracetate, amino phosphonates such as ethylenediaminetetrakis (methylene-phosphonates), polyfunctionally-substituted aromatic chelating agents and mixtures thereof.

Another suitable odor control agent for use herein is a buffer system, such as citric acid and sodium bicarbonate, sodium phosphate and sorbic acid buffer systems. Also, buffer systems having a pH of from 7 to 10 may be useful herein.

In addition, alternative odor control agents useful herein can be ion exchange resins.

This invention also contemplates the use of masking agents, such as perfumes, which can be used in place of or in combination with the more traditional odor control agents described above.

Antimicrobial agents such as chitosan and chitin-based polymers can be effective in combating malodors by preventing or retarding the growth of bacteria. As used herein, “antimicrobial” includes sequestering or immobilizing microorganisms such that their numbers within a suspension medium are reduced, even though the microorganisms may not be killed. Two examples of an antimicrobial include: chitosan VNS-608, commercially available from Vanson Chemical Company, Inc., Redmond, Wash. and carboxymethylcellulose CMC-7H3SXF, commercially available from Aqualon Oil Field Chemicals, a Division of Hercules, Inc., Houston, Tex.

Still another suitable odor control agent is peroxyacid, such as phthalimido and phthalimido peroxyalkanoic acids. A desirable peroxyacid is e-phthalimido peroxyhexanoic acid (PAP) which is commercially available from AUSIMONT and sold under the name PAP, or EURECO 0 (in granule form), or EURECO WKC (in wet granule form), or EURECO HC (in powdered active form).

The odor control substance 34 can be physically embedded in the material forming the disposal member 26. For example, the odor control substance 34 can be inserted into a film substrate such that as the disposal member 26 is formed, the odor control substance 34 will be an integral part thereof. Alternatively, the odor control substance 34 can be attached, printed, coated, slot coated, painted or applied onto the inner or outer surface of the disposal member 26. The odor control substance 34 can be an integral part of the disposal member 26 and may not be easily separated therefrom. An example of a commercially available product that contains an odor control substance is Bio-Fresh® poly bags. Bio-Fresh®) poly bags are manufactured by Active Pack International, Inc., having an office at 3175 Commercial Avenue, Northbrook, Ill., 60062.

A gelling agent can also be incorporated into the absorbent article 10 to slow down or stop the flow of excess fluid from the soiled absorbent article 10. Examples of suitable gelling agents include clequat polymers, UCARE polymers, chitosan, superabsorbent particles, absorbent polymers, etc. These gelling agents may be used alone or in combination with other agents. The gelling agents can be positioned on the disposal member 26 as a coating, be added to the disposal member 26 as a separate component or be physically embedded within the disposal member 26. U.S. Patent Application Publication 2003/0124936 A1 entitled: “ABSORBENT ARTICLE WITH FLUID TREATMENT AGENT” published Jul. 3, 2003 is representative of such a gelling agent being used in an absorbent article.

Still referring to FIGS. 1-3, the absorbent article 10 is shown having a garment adhesive 36 secured to the exterior surface of the baffle 16 and to the exterior surface of the disposal member 26. The garment adhesive 36 is optional and functions to secure the absorbent article 10 to the inside surface of the wearer's underpants. Usually, small absorbent articles like pantyliners, feminine pads, adult incontinent pads and sanitary napkins utilize one or more strips or areas of garment adhesive 36. The garment adhesive 36 can be a hot or cold melt adhesive. In FIGS. 1-3, the garment adhesive 36 is depicted as a single strip that runs parallel to the central longitudinal axis X-X, from the first end 22 to the second end 24. However, the entire length L of the absorbent article 10 does not have to be covered by the garment adhesive 36. In addition, the garment adhesive 36 does not have to be a continuous line, strip or area. The width of the garment adhesive 36 is shown as being less than the width W of the absorbent article 10. However, it should be noted that the width of the garment adhesive 36, like the length and thickness of the garment adhesive 36, can vary to suit one's particular needs. The garment adhesive 36 is shown covering about 35% of the outer surface of the absorbent article 10 but it could cover more or less of the outer surface. The garment adhesive 36 will usually cover from between about 25% to 100% of the outer surface of the absorbent article 10. Desirably, the garment adhesive 36 will cover from between about 30% to about 90% of the outer surface of the absorbent article 10.

In FIG. 1, one will notice that the garment adhesive 36 is situated on the baffle 16 as well as on the exterior surface of the disposal member 26. The garment adhesive 36 secured to the exterior surface of the disposal member 26 can aid in holding the inverted pocket 28 onto the rolled or folded absorbent article 10, as will be explained in more detail below.

Once the user of the absorbent article 10 has worn the article 10 for its intended purpose and for a given period of time, he or she will need to change or remove the article 10. The worn absorbent article 10 may have been insulted with body fluid and this soiled article 10 may need to be replaced. When this time comes, the user will remove the soiled absorbent article 10 from his or her crotch and discard the article 10. A common means of disposing of a used and soiled absorbent article is to flush it down a toilet if it is small in size or to place it in a toilet pail or other similar receptacle. However, this method of disposal is not always possible or warranted. As mentioned supra, there may be times when the user would rather wrap up the soiled absorbent article 10 and take it with them until it can be privately and discreetly disposed of. The absorbent article 10 of this invention permits this to be accomplished very easily.

Referring now to FIGS. 4-5, the soiled absorbent article 10 can be reduced in size by rolling it up into a small cylinder to obtain a compact article. In FIG. 4, the absorbent article 10 is rolled up. By “rolled” it is meant that one end of the absorbent article 10 is caused to move forward along a surface by revolving on an axis or by repeatedly turning over. In FIG. 4, the first end 22 of the absorbent article 10 is radially wound toward the second end 24 with the cover 12 being the upper surface and the baffle 16 being the lower surface. Once the absorbent article 10 is substantially rolled up into a cylinder 38, the pocket 28 is inverted or turned inside out and flipped or extended over and around the outer surface of the cylinder 38. The pocket 28 is of sufficient size to receive and enclose a substantial portion of the absorbent article 10. Desirably, the pocket 28 will be capable of receiving and enclosing the entire absorbent 14 of the absorbent article 10. This configuration is depicted in FIG. 5. One will notice that the open portion 32 has expanded to receive the absorbent article 10 and then retracts to a rather small slit. The disposal member 26, because of its retractable or contractible properties, is able to retain the absorbent article 10 in a substantially reduced or compact configuration. The garment adhesive 36, if present, will also assist the disposal member 26 in keeping the absorbent article 10 in a compact condition. The odor control substance 34, integrally formed in or on the disposal member 26, will ensure that no appreciable amount of malodors will exit from the soiled absorbent article 10.

It should be noted that the reduced or compact configuration of the absorbent article 10 can be maintained by utilizing a fastening mechanism to close the open portion 32 and seal the disposal member 26 once the soiled absorbent article 10 is contained therein. Such a fastening mechanism can include: a tape tab, a hook, micro hooks, a hook and loop fasteners, adhesive, etc. A commonly available hook and loop fastener is VELCRO®. VELCRO® is a registered trademark of Velcro USA, Inc., having an office at 406 Brown Avenue, Manchester, N.H. 03103.

Turning now to FIG. 6, the absorbent article 10 can also be made more compact by folding it one or more times upon itself. In FIG. 6, the absorbent article 10 has been folded in half such that the first end 22 is positioned over the second end 24. By “folding” it is meant that the absorbent article 10 is bent over or doubled up so that one part lies on another part. In the shown folded position, the soiled cover 12 and absorbent 14 are partially sheltered by the liquid-impermeable baffle 16. The pocket 28 is again inverted or turned inside out and flipped over the first end 22 such that a substantial portion of the absorbent article 10 is received in the open portion 32 and enclosed within the pocket 28. The folded and partially enclosed absorbent article 10 can now be inserted into the wearer's purse, into a pocket of an overcoat, into a hand bag, into a paper or plastic bag, or into some other enclosure and be removed and properly disposed of at a later time.

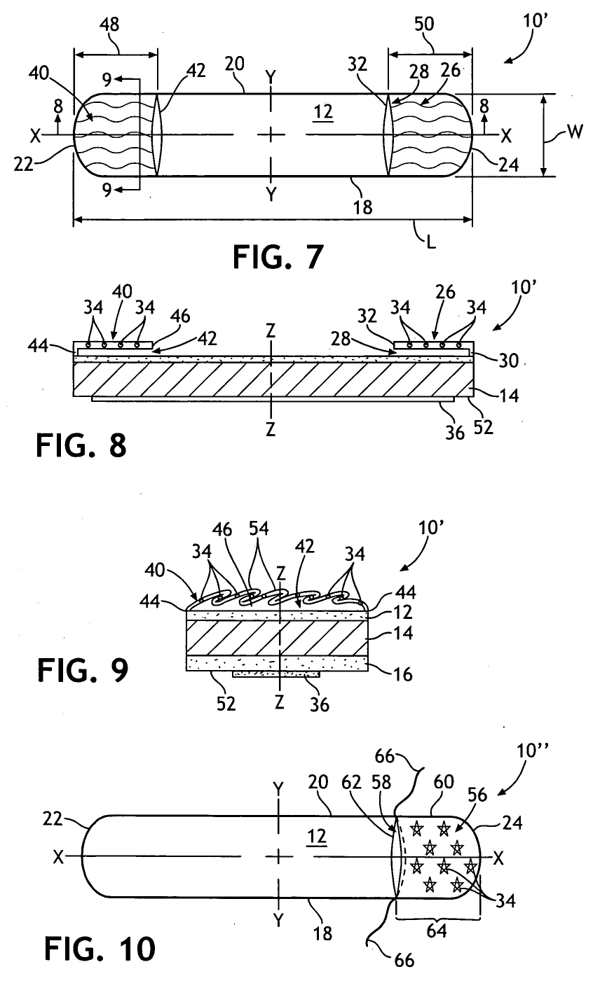

Referring now to FIGS. 7-9, an alternative embodiment of an absorbent article 10′ is shown having a pair of disposal members 26 and 40 formed from a material having an odor control substance 34 integrally formed therein. Each of the disposal members 26 and 40 forms a pocket, 28 and 42 respectively, positioned adjacent to one of the ends, 24 or 22 respectively, of the absorbent article 10′. The pocket 28 has a fixed portion 30 and an open portion 32, while the pocket 42 has a fixed portion 44 and an open portion 46. The fixed and open portions, 44 and 46 respectively, can be similar or identical to the fixed and open portions, 30 and 32 respectively, described above with reference to the pocket 28. In FIG. 7, the disposal members 26 and 40 are secured to the cover 12 and the pockets 28 and 42 extend over a limited portion of the cover 12. In this arrangement, the limited portions 48 and 50 of the cover 12 are positioned between the disposal members, 26 and 40 respectively, and the absorbent 14, see FIG. 8. Alternatively, each fixed portion 30 and 44 of the pockets, 28 and 42 respectively, can be secured to the absorbent 14 and the pockets 28 and 42 can extend over a limited portion, 48 and 50 respectively, of the cover 12. In this arrangement, the limited portions 48 and 50 of the cover 12 are again positioned between the disposal members, 26 and 40 respectively, and the absorbent 14.

In FIGS. 7-9, the cover 12 has an outer periphery and the fixed portions 30 and 44 of the pockets, 28 and 42 respectively, are secured to the periphery of the cover 12. Alternatively, the absorbent 14 also has an outer periphery and the fixed portions 30 and 44 of the pockets, 28 and 42 respectively, can be secured to the periphery of the absorbent 14. One will also notice that the absorbent article 10′ does not have a baffle. Instead, the absorbent 14 has a lower surface 52, see FIG. 8, that can be treated, coated, painted or conditioned to take on the characteristic of being liquid-impermeable. Alternatively, a portion of the absorbent 14 can be manufactured to have a high density such that it is almost impossible for body fluid to pass therethrough. Desirably, it will be the lower portion of the absorbent 14 that is so conditioned to have the higher density. Secured to a portion of the lower surface 52 is a garment adhesive 36. The function and makeup of the garment adhesive 36 is identical to that explained above for FIGS. 1-3.

Turning now to FIG. 9, one will notice that the disposal member 40 is configured to have a plurality of pleats 54. Each pleat 54 is formed by folding or doubling the material onto itself. The function of the pleats 54 is that they can increase the dimension of the material from which the disposal member 40 is formed as they unfold thereby allowing the pocket 42 to become much larger. This ability to increase the size for the pocket 42 will enhance the ability of the user to roll or fold up the absorbent article 10′ and tuck it into the open portion 46 such that it will be substantially enclosed by the disposal member 40. Once the pleats 54 have fully expanded, the material will not be able to expand outward an additional amount, and this will assure that the compacted absorbent article 10′ retained within the pocket 42 will be retained in a rather compacted state.

Referring now to FIG. 10, another embodiment of an absorbent article 10″ is shown having a single disposal member 56 secured to the cover 12. The disposal member 56 is positioned adjacent to the second end 24 of the absorbent article 10″. The disposal member 56 extends over a limited portion 64 of the cover 12 such that the limited portion 64 of the cover 12 is positioned between the disposal member 56 and the absorbent 14 (not shown). In other words, the disposal member 56 is initially situated over a portion of the upper surface of the cover 12 and does not need to be inverted in order to enclose a substantial portion of the absorbent article 10″. The disposal member 56 forms a pocket 58 having a fixed portion 60 and an open portion 62. The fixed and open portions, 60 and 62 respectively, can be similar or identical to the fixed and open portions, 30 and 32 respectively, described above with reference to the pocket 28 when referring to FIGS. 1-3.

Another difference depicted in the embodiment shown in FIG. 10, is that the odor control substance 34 is printed on the upper surface of the disposal member 56 in the form of multiple characters, for example stars. It should be recognized that any design or arrangement within one's imagination can be used for the profile of the odor control substance 34. Examples include, but are not limited to, circles, squares, triangles, letters, printed word(s), symbols, or graphics of any kind. Graphics of human figures, spiritual beings, manmade objects, movable objects such as a cloud, an animal, an angel, etc. can be used. Likewise, combinations of letters, symbols and/or graphics can also be used. The multiple spaced apart characters, i.e. stars, shown in FIG. 10, can add a decorative flare and/or color to the absorbent article 10″. The multiple spaced apart characters can also be printed on in a variety of colors to attract the attention of the user. The printed symbols of the odor control substance 34 can be used to inform the ultimate consumer that that particular surface of the absorbent article 10″ is designed to be placed adjacent to his or her skin. This will help assure that the absorbent article 10″ is properly positioned on the human body.

FIG. 10 also has a draw string 66 which is positioned across the open portion 62 of the pocket 58 and attached to the disposal member 56. The draw string 66 can be pulled tight once the rolled or folded soiled absorbent article 10″ is enclosed by the pocket 58. The opposite ends of the draw string 66 can then be tied together to ensure that the soiled absorbent article 10″ is retained in a compacted configuration.

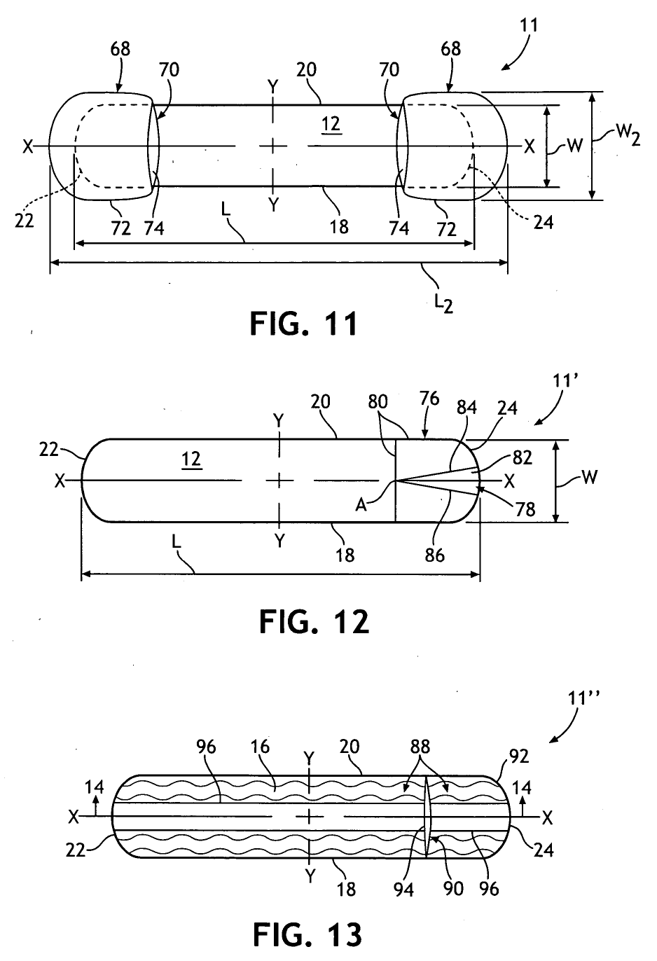

Moving on to FIG. 11, still another embodiment of an absorbent article 11 is shown having a pair of disposal members 68 formed from a material having an odor control substance integrally formed therewith. Each of the disposal members 68 extends beyond one of the opposite ends 22 and 24 of the absorbent article 11. Each of the disposal members 68 forms a pocket 70 having a fixed portion 72 and an open portion 74. The fixed and open portions, 72 and 74 respectively, can be similar or identical to the fixed and open portions, 30 and 32 respectively, described above with reference to the pocket 26 shown in FIGS. 1-3. Each of the disposal members 68 is secured over the cover 12 and each can be secured to the cover 12, the absorbent 14 or to the baffle 16. Each of the disposal members 68 is larger in size than those described earlier. In FIG. 11, each of the disposal members 68 extends beyond one of the ends 22 and 24 such that they increase the length L of the absorbent article 11 from the initial length L to a longer length L₂. Likewise, each of the disposal members 68 can extend beyond the width W of the absorbent article 11 such that they increase the width W of the absorbent article 11 from the initial width W to a wider width W₂. It is not necessary that the disposal members 68 extend both the length and the width dimensions of the absorbent article 11 but can extend only one of the dimensions, if desired.

If the disposal members 68 are formed from a liquid-impermeable material, the enlarged configuration of the pair of disposal members 68 can act as shields and further prevent any body fluid that may contact the disposal members 68 from contacting the user's skin. In essence, the disposal members 68 can increase the overall size of the absorbent article 11 and help to prevent leakage from occurring while the absorbent article 11 is being worn. The actual size and configuration of the disposal members 68 can be varied to meet one's individual needs.

Alternatively, an absorbent article 11 can be assembled and then one or two disposal members 68 can be secured thereto. If the absorbent article 11 is manufactured such that it has a peripheral seal and an outlying fringe (not shown), the disposal member(s) 68 can be attached or secured to the seal or to the fringe. The disposal member(s) 68 can further be an extension of the cover 12, the absorbent 14 or the baffle 16. The pair of disposal members 68 can also be constructed from a different material than the cover 12 or the baffle 16, if desired.

Referring to FIG. 12, still another embodiment of an absorbent article 11′ is shown having a disposal member 76. The disposal member 76 is secured so as to lie over and across the width W of the cover 12. The disposal member 76 is positioned adjacent to the second end 24 of the absorbent article 11′. The disposal member 76 forms a pocket 78 having a fixed portion 80 and an open portion 82. The fixed and open portions, 80 and 82 respectively, are different from those described above in that the open portion 82 has a V-shaped opening formed by two intersecting lines 84 and 86. Each of the lines 84 and 86 is aligned at an angle of from between about 1 degree to about 45 degrees to the central longitudinal axis X-X. The intersecting lines 84 and 86 converge at a point (A) where the pocket 78 is affixed to the cover 12. Point A is shown being distally spaced apart from the second end 24 but it could be aligned close to the second end 24, if desired. The V-shaped opening gets larger as the open portion 82 moves away from point A. It should be noted that point A can be located on the outer periphery of the second end 24 and the open portion 82 can be rotated 180 degrees from that shown in FIG. 12, if desired. As depicted, the fixed portion 80 of the pocket 78 tracks the outer periphery of the cover 12 around the second end 24 except for where the open portion 82 resides.

Once the absorbent article 11′ has been soiled and removed from the human body, it is rolled or folded starting approximate the first end 22 and advancing toward the second end 24. The compacted absorbent article 11′ is then tucked into the open portion 82 by expanding the edges coaxially aligned with the intersecting lines 84 and 86. This action allows the rolled or folded absorbent article 11′ to enter the open portion 82 and be substantially enclosed in the pocket 78. The V-shaped opening is allowed to contract back to or towards its original size once the absorbent article 11′ is retained within the pocket 78.

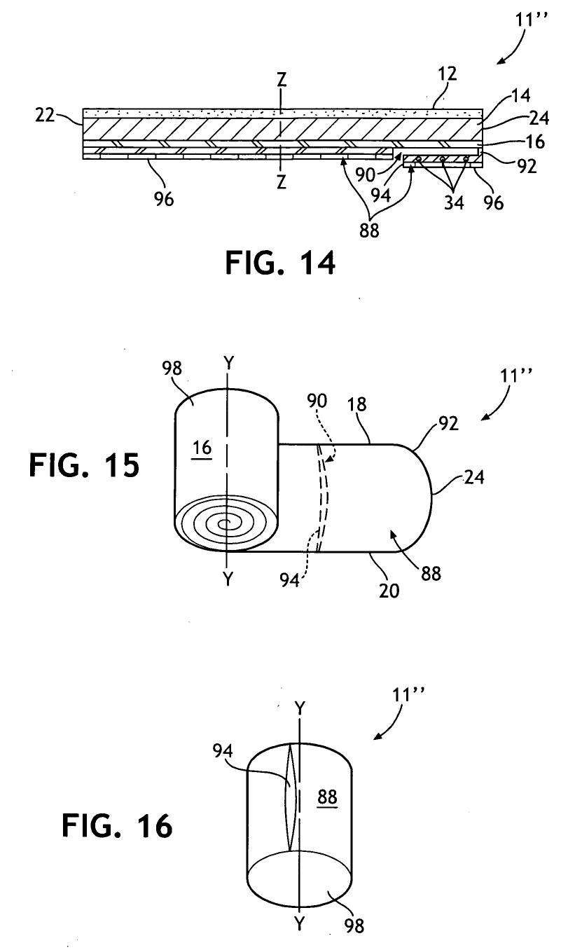

Referring to FIGS. 13-16, still another embodiment of an absorbent article 11″ is shown having a disposal member 88 formed completely over the baffle 16 and having an odor control substance integrally formed therewith. The disposal member 88 forms a pocket 90 having a fixed portion 92 and an open portion 94. The fixed portion 92 is coterminuous with the outer periphery of the baffle 16. The disposal member 88 completely covers or over lapses the baffle 16 except for the narrow slit which forms the open portion 94. A strip of garment adhesive 96 is secured to the lower surface of the disposal member 88 and extends from the first end 22 to the second end 24 of the absorbent article 11″. The garment adhesive 96 is optional and may not be needed on all absorbent articles. As stated above with reference to FIGS. 1-3, the length, width and thickness of the garment adhesive 96 can be varied to suit one's particular needs and requirements. Even though the embodiment depicted in FIGS. 13-16 requires additional material to form the disposal member 88, the fact that the disposal member 88 is a single layer having essentially the same outline as the cover 12, the absorbent 14, and the baffle 16, it may be easier to manufacture. This ease in manufacturing may offset the expense of using the added material.

Lastly, turning to FIGS. 15 and 16, the soiled absorbent article 11″ can be reduced in size by rolling it up into a small cylinder to obtain a compact article. In FIG. 15, the absorbent article 11″ is rolled up wherein the first end 22 of the absorbent article 11″ is radially wound toward the second end 24 with the cover 12 being the upper surface and the baffle 16 being the lower surface. Once the absorbent article 11″ is substantially rolled up into a cylinder 98, the pocket 90 is inverted or turned inside out and flipped or extended over and around the outer surface of the cylinder 98. The pocket 90 is of sufficient size to receive and enclose a substantial portion of the absorbent article 11″. Desirably, the pocket 90 will be capable of receiving and enclosing the entire absorbent 14 of the absorbent article 11″. This configuration is depicted in FIG. 16. One will notice that the open portion 94 has expanded to receive the absorbent article 11″ and then retracts to a rather small slit. The disposal member 88, because of its retractable or contractible properties, is able to retain the absorbent article 11″ in a substantially reduced or compact configuration. The garment adhesive 96, if present, will also assist the disposal member 88 in keeping the absorbent article 11″ in a compact condition. The odor control substance 34, integrally formed in or on the disposal member 88, will ensure that no appreciable amount of malodors will exit from the soiled absorbent article 11″.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. An absorbent article comprising:
a) a cover;
b) an absorbent positioned adjacent to said cover; and
c) a disposal member configured as a pocket having a fixed portion and an open portion, said fixed portion secured to at least one of said cover and said absorbent, said disposal member being formed from a material having an odor control substance integrally formed therewith, and said open portion of said pocket being capable of receiving and enclosing a substantial portion of said absorbent article after it has been reduced in overall size.

2. The absorbent article of claim 1 wherein said absorbent article has a longitudinal centerline and a pair of spaced apart ends, said disposal member is secured about one of said ends with said open portion of said pocket being aligned perpendicular to said longitudinal centerline, and said absorbent article is rolled up after being soiled by a body fluid and at least partially inserted into said open portion of said pocket for convenient disposal.

3. The absorbent article of claim 2 wherein said fixed portion is secured to said cover and said pocket extends over a limited portion of said cover such that said limited portion of said cover is positioned between said disposal member and said absorbent.

4. The absorbent article of claim 2 wherein said fixed portion is secured to said absorbent and said pocket extends over a limited portion of said cover such that said limited portion of said cover is positioned between said disposal member and said absorbent.

5. The absorbent article of claim 1 wherein said absorbent includes a liquid permeable region and a liquid-impermeable region.

6. The absorbent article of claim 1 wherein said odor control substance is selected from a group consisting of a zeolite, an activated carbon, clay, a molecular sieve, a peroxyacid, a carbonate, a bicarbonate, a perfume, a starch, a superabsorbent or a mixture thereof.

7. The absorbent article of claim 1 wherein said odor control substance is selected from a group consisting of an antimicrobial agent, a chelating agent or a mixture thereof.

8. The absorbent article of claim 1 wherein said cover has a periphery and said fixed portion of said disposal member is secured to the periphery of said cover.

9. The absorbent article of claim 1 wherein said absorbent has a periphery and said fixed portion of said disposal member is secured to the periphery of said absorbent.

10. An absorbent article comprising:
a) a cover;
b) a baffle;
c) an absorbent positioned between said cover and said baffle; and
d) a disposal member configured as a pocket having a fixed portion and an open portion, said fixed portion secured to at least one of said cover, absorbent and baffle, said disposal member being formed from a resiliently extensible material having an odor control substance integrally formed therewith, and said open portion of said pocket being capable of receiving and enclosing a substantial portion of said absorbent article after it has been reduced in overall size.

11. The absorbent article of claim 10 wherein said odor control substance is printed on said disposal member.

12. The absorbent article of claim 11 wherein said odor control substance is printed in the form of multiple characters.

13. The absorbent article of claim 10 wherein a draw string is attached to said disposal member and can be pulled tight to close said open portion once said absorbent article is enclosed within said pocket.

14. The absorbent article of claim 10 wherein a pair of disposal members are secured to said cover and each is positioned adjacent to an end of said absorbent article, and said pair of disposal members increases the overall length of said absorbent article.

15. The absorbent article of claim 10 wherein a pair of disposal members are secured to said cover and each is positioned adjacent to an end of said absorbent article, and said pair of disposal members increases the overall width of said absorbent article.

16. An absorbent article comprising:
a) a liquid permeable cover;
b) a liquid-impermeable baffle;
c) an absorbent positioned between said cover and said baffle; and
d) a disposal member configured as a pocket having a fixed portion and an open portion, said fixed portion secured to at least one of said cover, absorbent and baffle, said disposal member being formed from a resiliently extensible material having an odor control substance integrally formed therewith, said odor control substance capable of limiting any malodors exiting from said absorbent, said disposal member extending over a limited portion of said cover such that said limited portion of said cover is positioned between said disposal member and said absorbent, and said open portion of said pocket being capable of receiving and enclosing said absorbent article after it has been reduced in overall size.

17. The absorbent article of claim 16 wherein said absorbent article is reduced in size by rolling it up upon itself.

18. The absorbent article of claim 16 wherein said absorbent article is reduced in size by folding it at least once upon itself.

19. The absorbent article of claim 16 wherein said absorbent article has a longitudinal centerline and a pair of spaced apart ends, said disposal member is secured about one of said ends with said open portion of said pocket being aligned perpendicular to said longitudinal centerline, and said absorbent article is rolled up and at least partially inserted into said open portion of said pocket for convenient disposal.

20. The absorbent article of claim 16 wherein said absorbent article has a longitudinal centerline and a pair of spaced apart ends, said disposal member is secured about one of said ends and said open portion of said pocket is a V-shaped opening formed by two intersecting lines, each line aligned at an angle of from between about 1 to about 45 degrees to said longitudinal centerline, and said absorbent article is rolled up and at least partially inserted into said open portion of said pocket for convenient disposal.

21. The absorbent article of claim 16 wherein said cover has a predetermined surface area and said disposal member extends over less than about 30% of said predetermined surface area.

22. The absorbent article of claim 16 wherein said absorbent article has a longitudinal centerline and a pair of spaced apart ends, said disposal member is secured to said baffle and is coterminuous therewith, and said disposal member has a pair of open portions formed therein with each being located adjacent to one of said pair of spaced apart ends.

23. The absorbent article of claim 16 wherein said disposable member is liquid permeable.

24. The absorbent article of claim 16 wherein a pair of disposal members is secure to at least one of said cover, absorbent and baffle and each is positioned adjacent to an end of said absorbent article, and said pair of disposal members increases the overall length of said absorbent article.
